(19) United States
(12) Patent Application Publication
(10) Pub. No.: US 2012/0209011 A1
(43) Pub. Date: Aug. 16, 2012

(54) NOVEL PROCESS FOR THE PREPARATION OF PROSTAGLANDINS AND INTERMEDIATES THEREOF

(76) Inventors: Chandrashekar Aswathanarayanappa, Bangalore (IN); Pullela Venkata Srinivas, Bangalore (IN); Divya Kangath, Bangalore (IN); Thilak Gregory Soundararajan, Trichy (IN); Anegondi Sreenivasa Prasad, Hydrabad (IN); Suriyan Masinaickenpatty Raghavendran, Tiruchirapalli (IN)

(21) Appl. No.: 13/503,192

(22) PCT Filed: Dec. 21, 2009
(86) PCT No.: PCT/IN09/00730

§ 371 Date: Apr. 20, 2012

(30) Foreign Application Priority Data
Nov. 5, 2009 (CH) 0269/CHE/2009

(51) Int. Cl. C07C 405/00; C07D 307/935

(52) U.S. Cl. 549/311; 549/465; 562/503; 560/121; 564/139

(57) ABSTRACT

This invention relates to novel process for the preparation of prostaglandin compounds having formula (K),

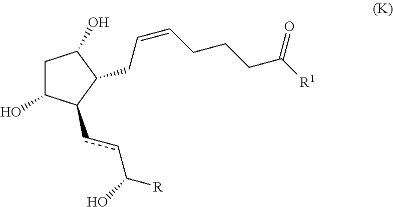

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H; and dashed lines represents a double bond or a single bond, is disclosed. Novel intermediates are also disclosed.

### FIELD OF THE INVENTION

The present invention relates to a novel process for the preparation of prostaglandins and prostaglandin analogues. The present invention further relates to novel synthetic intermediates that are used in the preparation of prostaglandins and prostaglandin analogues.

### BACKGROUND AND PRIOR ART OF THE INVENTION

Ocular hypertension and glaucoma can be effectively controlled using prostaglandin related drugs. Glaucoma is an eye disorder characterized by increased intraocular pressure and gradual loss of the visual field. An abnormally high intraocular pressure is commonly known to be detrimental to the eye, and there are clear indications that, in glaucoma patients, this probably is the most important factor causing degenerative changes in the retina. Unless treated successfully glaucoma will lead to blindness sooner or later, its course towards that stage is typically slow with progressive loss of the vision.

WO 90/02553 describes the use of prostaglandin derivatives of PGA, PGB, PGD, PGE and PGF, in which the omega chain has been modified with the common feature of containing a ring structure, for the treatment of glaucoma or ocular hypertension. The invention relates also to ophthalmic compositions, containing an active amount of these prostaglandin derivatives, and the manufacture of such compositions. WO 93/00329 describes the novel process for the preparation of 13,14-dihydro-15(R)-17-phenyl-18,19,20-trinor-PGF2α esters.

The present invention is to provide a novel process for the preparation of prostaglandins and prostaglandin analogues in good yield, in large

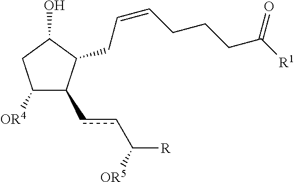

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H

### DETAILED DESCRIPTION

The present invention is in relation to a process for preparing compound of formula;

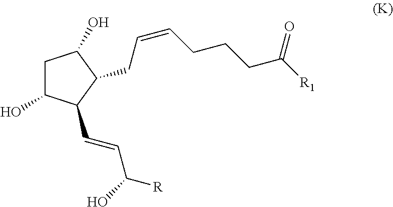

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-amounts and with desired purity. The present process minimizes the formation of impurities

Further, this invention provides process for the preparation of novel intermediates used in the preparation of prostaglandins and their analogues.

### OBJECTIVES OF THE INVENTION

First objective of the present invention is to provide a process for preparation of prostaglandins.

Second objective of the present invention is to provide a process for preparation of prostaglandins and also a process to prepare its intermediates.

### STATEMENT OF THE INVENTION

Accordingly, the present invention is in relation to a process for preparing compound of formula;

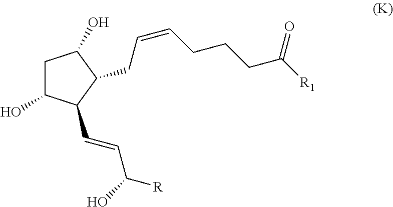

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H, and dashed lines ( [symbol] ) represents a double bond or a single bond comprises; a). reacting compound of formula ‘I’ with haloalkane or ethylamine,

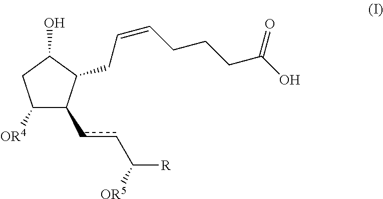

Wherein, R described as above, dashed line represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H to form compound of formula ‘J’ and,

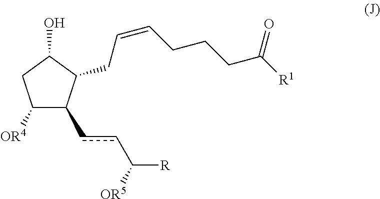

Wherein, R, R¹, dashed line, R⁴ and R⁵ as described above; and b). deprotecting compound of formula ‘J’; A compound of formula;

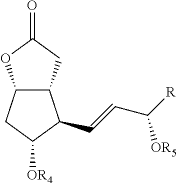

Wherein R described as above, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H; a compound of formula;

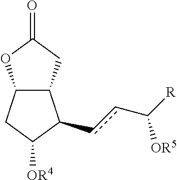

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H; a compound of formula;

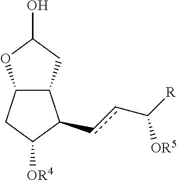

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H; a compound of formula;

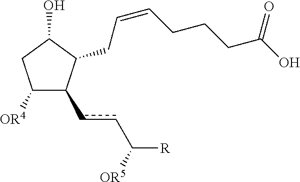

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H; and a compound
C₆ alkyl, halo and CF₃; and R¹ is selected from OR³ and NHR³ wherein R³ is C₁-C₆ alkyl, H, and dashed lines ( [symbol] ) represents a double bond or a single bond comprises; a). reacting compound of formula ‘I’ with haloalkane or ethylamine,

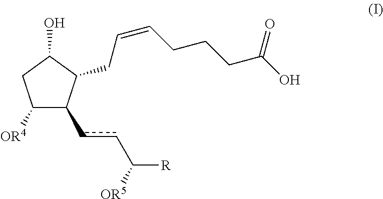

Wherein, R described as above, dashed line represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H to form compound of formula ‘J’ and,

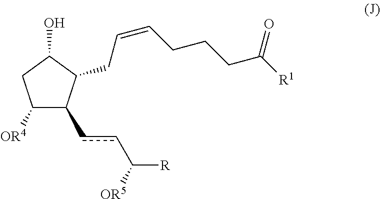

Wherein, R, R¹, dashed line, R⁴ and R⁵ as described above; and b). deprotecting compound of formula ‘J’.

In another embodiment of the invention deprotection is done using cerium (III) chloride heptahydrate and sodium iodide in the presence of organic solvent.

In yet another embodiment of the invention organic solvent is selected from a group comprising acetonitrile, ethanol, methanol, acetone and isopropyl alcohol.

In still another embodiment of the invention the compound K is any one of following compound;

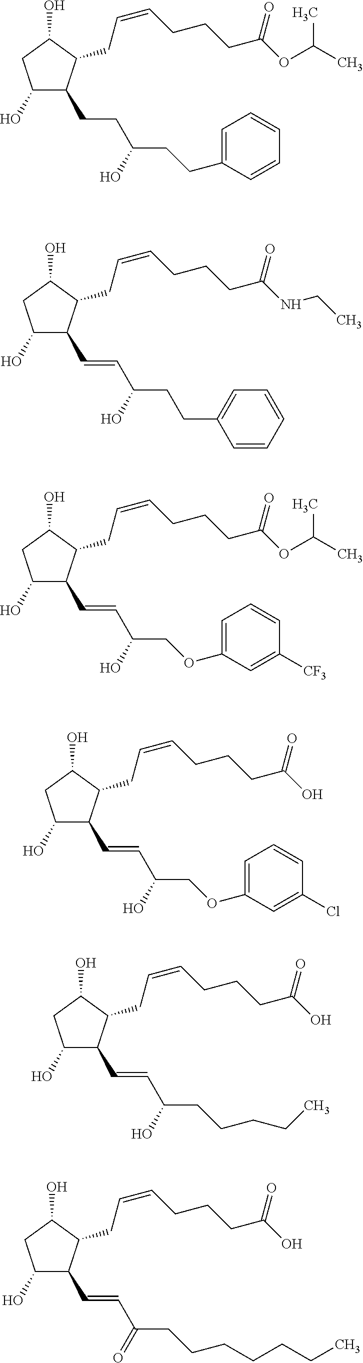

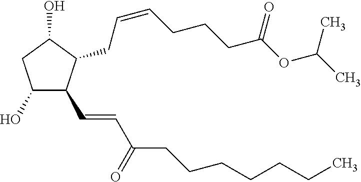

In still another embodiment of the invention process for the preparation of compound of formula ‘I’ comprises;

a). Reacting compound of formula ‘A’

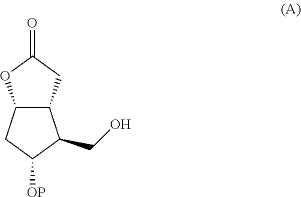

with dimethylsulphoxide, oxalylchloride and triethylamine in the presence of organic solvent to get compound of formula B′, wherein P is selected from the group consisting of COX; in which X represents C₁ to C₆ alkyl, C₆-C₁₀ aryl which may be substituted or unsubstituted with one to three substituents independently selected from the group consisting of halo, C₁ to C₆ alkyl, unsubstituted C₆ to C₁₀ aryl,

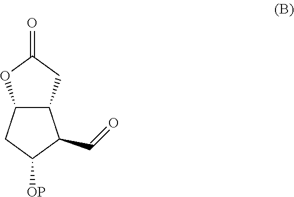

b). reacting compound of formula B′ with

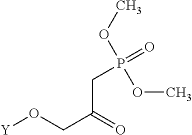

Wherein Y is selected from the group consisting of alkyl, aryl wherein aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃ in the presence of organic solvent to form compound of formula ‘C’

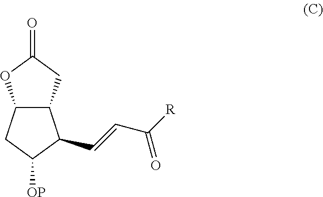

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃ and P is as described above,
c). by selective reduction of compound ‘C’ using Borane N,N′-diethylaniline complex in the presence of Corey catalyst to compound ‘D’,

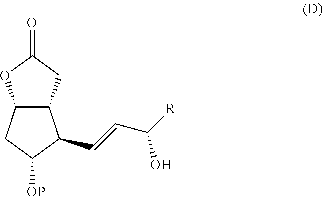

Wherein P and R are as described above,

d). deprotecting compound of formula D using base in organic solvent to get compound of formula E,

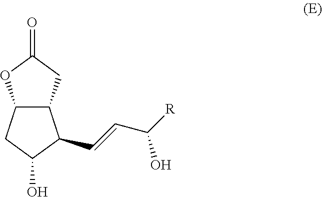

e). hydroxyl groups of compound E further protected to form compound of formula F,

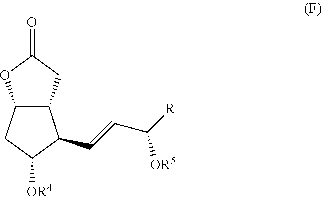

Wherein R described as above, R⁴ and R⁵ represents,

R⁴=R⁵=CH₂OCH₂CH₂CH₂CH₃; R⁴=H, R⁵=CH₂OCH₂CH₂CH₂CH₃;

### R⁴=CH₂OCH₂CH₂CH₂CH₃, R⁵=H

f). compound of formula F optionally hydrogenated using palladium on carbon in the presence of organic solvent, further reducing the oxo group of this compound reduced to compound of formula H using DIBAL-H in the presence of organic solvent,

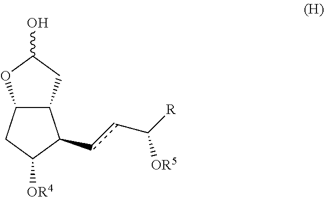

wherein the dashed line represents a double bond or a single bond; R, R⁴ and R⁵ represents as described above
g). compound of formula H further reacted with compound of formula PPh₃(CH₂)₄COOH in the presence of NaHMDS in organic solvent

In still another embodiment of the invention organic solvent is selected from a group comprising of alcohols, esters, tetrahydrofuran, pet ether, hexane, acetone and acetonitrile.

In still another embodiment of the invention said alcohols are selected from C₁ to C₄ alcohols.

In still another embodiment of the invention said esters are selected from ethyl acetate or butyl acetate.

In still another embodiment of the invention base is selected from potassium carbonate, sodium carbonate or sodium bi carbonate.

The present invention is in relation to a compound of formula;

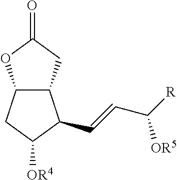

Wherein R described as above, R⁴ and R⁵ represents,

R⁴=R⁵=CH₂OCH₂CH₂CH₂CH₃; R⁴=H, R⁵=CH₂OCH₂CH₂CH₂CH₃;

### R⁴=CH₂OCH₂CH₂CH₂CH₃, R⁵=H

The present invention is in relation to a compound of formula;

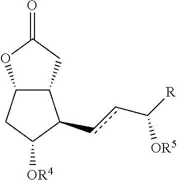

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H.

The present invention is in relation to a compound of formula;

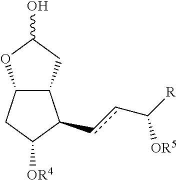

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H

The present invention is in relation to a compound of formula;

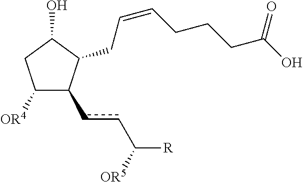

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H

The present invention is in relation to a compound

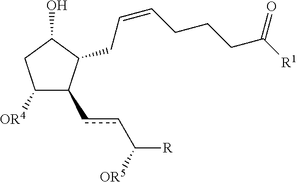

Wherein R described as above, dashed lines represents single or double bonds, R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H

The present invention provides a process for the preparation of prostaglandins and prostaglandin analogues. Ideally the synthetic route will be generally applicable to a variety of prostaglandin compounds and will provide high yields.

Accordingly, the present invention provides a process for the preparation of prostaglandins and prostaglandin analogues having the formula (K):

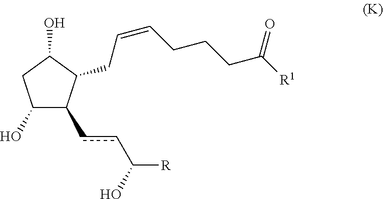

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H; and dashed lines represents a double bond or a single bond.

The following scheme 1 shows the synthesis of prostaglandins of formula (K) starting from Corey lactone.

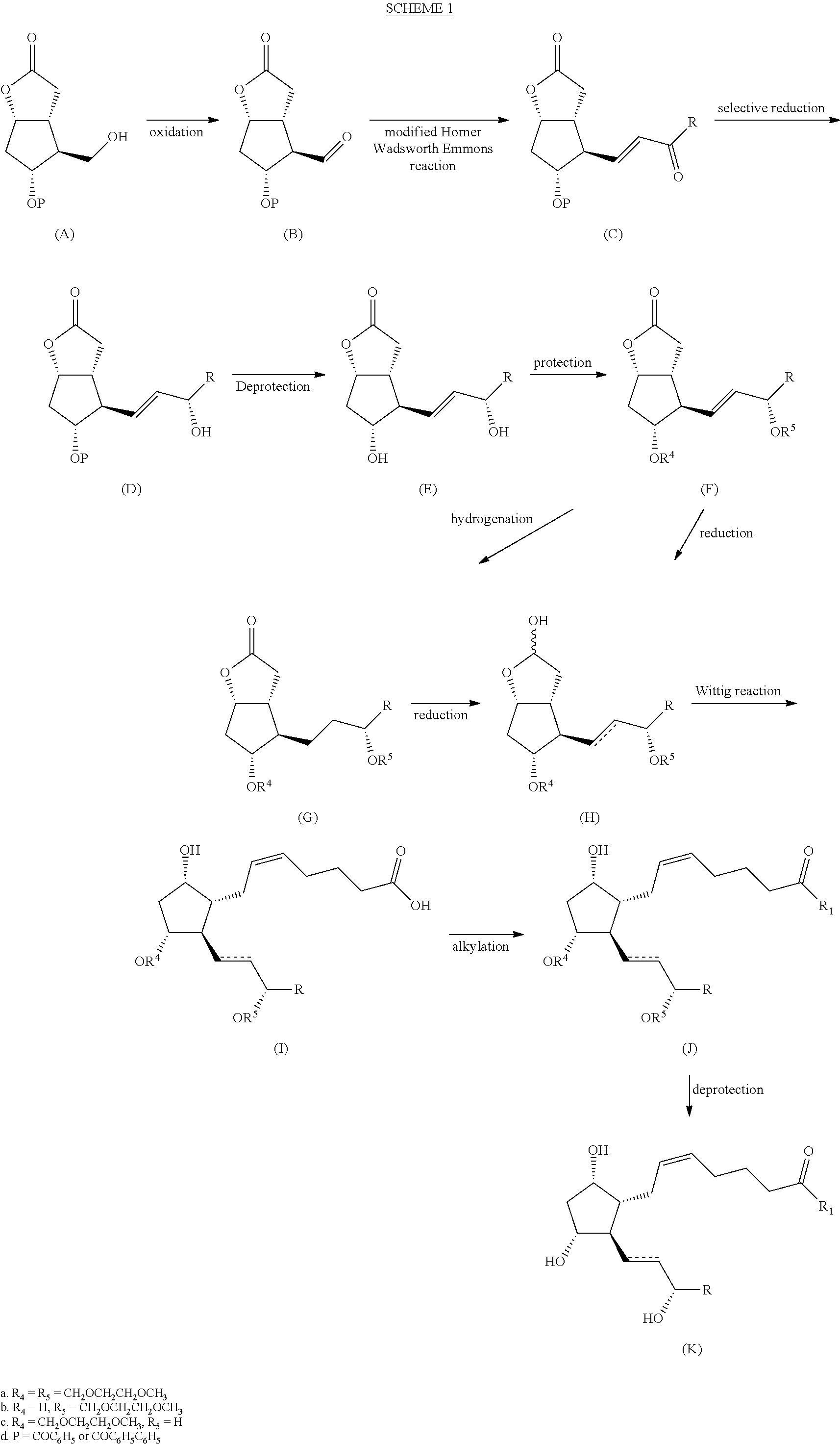

The present invention provides a process for the preparation of prostaglandins and prostaglandin analogues. Ideally the synthetic route will be generally applicable to a variety of prostaglandin compounds and will provide high yields.

Accordingly, the present invention provides a process for the preparation of prostaglandins and prostaglandin analogues having the formula (K):

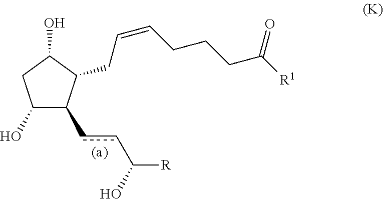

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H; and formula (a) represents a double bond or a single bond.

In one of the aspects of present invention, there is provided a process for the production of a compound of formula (B):

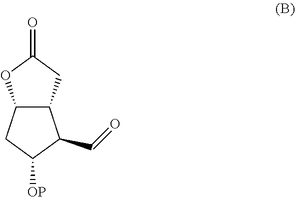

wherein P is selected from the group consisting of COX; wherein X represents C₁ to C₆ alkyl, C₆-C₁₀ aryl which may be un substituted or substituted with one to three substituents independently selected from the group consisting of halo, C₁ to C₆ alkyl, unsubstituted C₆ to C₁₀ aryl;
the process comprising subjecting a compound of formula (A)

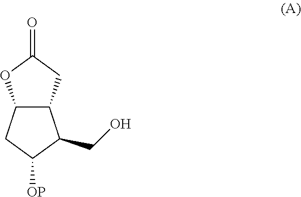

where P represents as described above
to an oxidation reaction using dimethylsulphoxide, oxalylchloride and triethylamine (Swern oxidation). Dimethylsulphoxide is activated by using oxalylchloride and dichloromethane as solvent. The aldehyde thus obtained (which is in dichloromethane) is taken for the next stage.

Advantageously, in the present method of oxidation of the compound of formula (A) using dimethylsulphoxide, oxalyl chloride and triethylamine (Swern oxidation), is a controllable reaction, minimizing the formation of acid. The aldehyde (B) in solution obtained in this step can be employed in the subsequent step without isolating aldehyde.

In a further aspect of present invention, there is provided a process for production of a compound of formula (D):

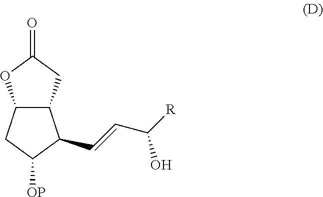

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and P represents as described above.
the process comprising subjecting a compound of formula (C)

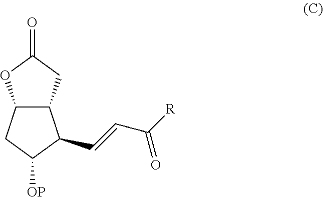

wherein P and R represent as described above.
to a selective reduction reaction using Borane N,N′-diethylaniline complex. The side chain oxo group (C) is reduced selectively to alcohol (D) by using Borane N,N′-diethylaniline complex in the presence of a chairal oxazaborolidine catalyst (“Corey catalyst”) to achieve desired isomer.

The advantage of the present method of selective reduction using Borane N,N′-diethylaniline complex in the presence of a chairal oxazaborolidine catalyst (“Corey catalyst”) is to achieve greater selectivity towards the production of major amount of desired isomer. Any undesired isomer which may be formed may be separated by chromatographic techniques such as flash column chromatography.

Thus, preferred reagent for the reduction of oxo (C) compound to alcohol (D) is borane N,N′-diethylailine complex in the presence of a chairal oxazaborolidine catalyst (“Corey catalyst”). The use of borane N,N′-diethylailine complex with a Corey catalyst is preferred because the reaction takes place with excellent selectivity. In fact, a marked improvement in selectivity is observed when compared with reaction using borane-dimethylsulphide complex.

According to another aspect of present invention, there is provided a process for the production of a compound of formula (F)

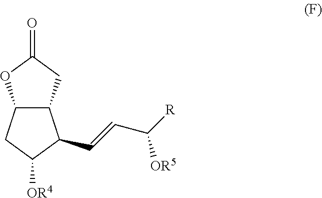

wherein R⁴ and R⁵ is alkoxyalkoxyalkyl, R⁴=R⁵=CH₂OCH₂CH₂OCH₃; R⁴=H, R⁵=CH₂OCH₂CH₂OCH₃; or R⁴=CH₂OCH₂CH₂OCH₃; R⁵=H; and R represents as described above
the process comprising protecting hydroxyl groups of a compound formula (E):

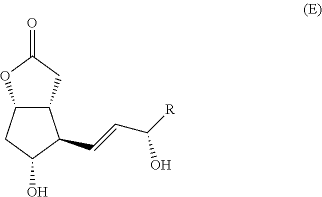

wherein R represents as described above.
the hydroxyl groups are protected using alkoxyalkoxyalkyl group. The hydroxyl groups of compound (E) are protected by using 2-methoxyethoxymethyl chloride (MEM chloride).

The use of alkoxyalkoxyalkyl protecting groups in the present process has a particular advantage compared with the prior art process employing benzoyl and para-phenylbenzoyl protecting groups because alkoxyalkoxyalkyl protecting groups are stable to the subsequent reduction reaction with e.g. DIBAL-H (diisobutylaluminium hydride). Alkoxyalkoxyalkyl protecting groups have further advantage in that they generally increase the lipophilic character of the molecule, so that their derivatives are readily soluble in organic solvents.

According to another aspect of present invention, there is provided a process for the production of a compound of formula (G):

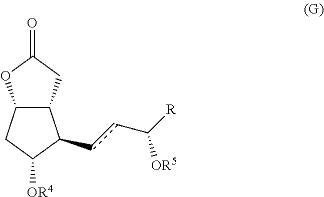

wherein the dashed line represents a double bond or a single bond; R, R⁴ and R⁵ represents as described above
the process comprising hydrogenating a compound formula (F) using palladium on carbon in the presence of ethyl acetate as solvent.

According to another aspect of present invention, there is provided a process for the production of a compound of formula (H):

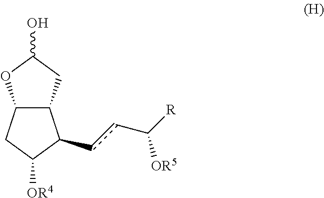

wherein the dashed line represents a double bond or a single bond; R, R⁴ and R⁵ represents as described above the process comprising reducing the lactone oxo group a compound formula (G) using DIBAL-H (diisobutylaluminium hydride) in the presence of tetrahydrofuran.

According to another aspect of present invention, there is provided a process for the production of a compound of formula (I):

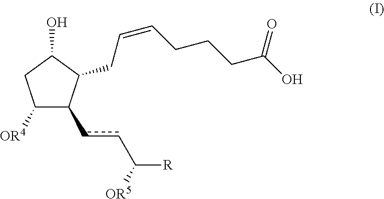

wherein the dashed line represents a double bond or a single bond; R, R⁴ and R⁵ represents as described above
the process comprising subjecting a compound of formula (H) to a Wittig reaction with (4-carboxybutyl)triphenylphosphonium bromide using sodium hexamethyldisilazane (NaHMDS) as a base and tetrahydrofuran as solvent.

The advantage of the present method of Wittig reaction using sodium hexamethyldisilazane (NaHMDS) is improvement in the yield compared to potassium-tert-butoxide. The other advantage of using alkoxyalkoxyalkyl protecting groups in the Wittig reaction is that the formation of desired cis-isomer is favored.

According to another aspect of present invention, there is provided a process for the production of a compound of formula (J):

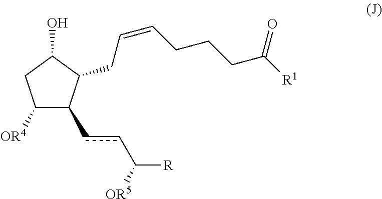

wherein the dashed line represents a double bond or a single bond; R, R⁴ and R⁵ represents as described above and R¹ is selected from OR³ and NHR³ wherein R³ is C₁-C₆ alkyl, H;
the process comprising subjecting a compound of formula (I) to reaction with an alkyl halide of formula, R′—X wherein R′ represents C₁ to C₆ alkyl groups or C₃ to C₈ cycloalkyl groups and X represents halogens such as chloro, bromo, or iodo, in the presence of DBU (1,8-Diazabicyclo[5.4.0]undec-7-ene) and acetone as solvent.

The ester thus obtained is converted into its amide in the presence of amines of formula R″—NH₂, wherein R″ represents C₁-C₆ alkyl, H;

According to another aspect of present invention, there is provided a process for the production of a compound of formula (K):

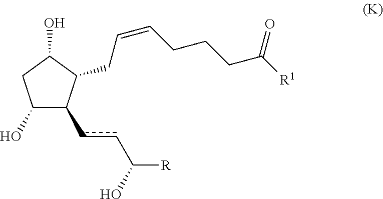

wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H; the dashed line represents a double bond or a single bond.

the process comprising deprotection of hydroxyl groups in compound of formula (J), by using cerium chloride and sodium iodide in the presence of acetonitrile as solvent at reflux temperatures.

The process of the present invention is particularly applicable for the production of prostaglandins and prostaglandin analogues. The process is particularly useful for the production of compounds selected the group consisting of

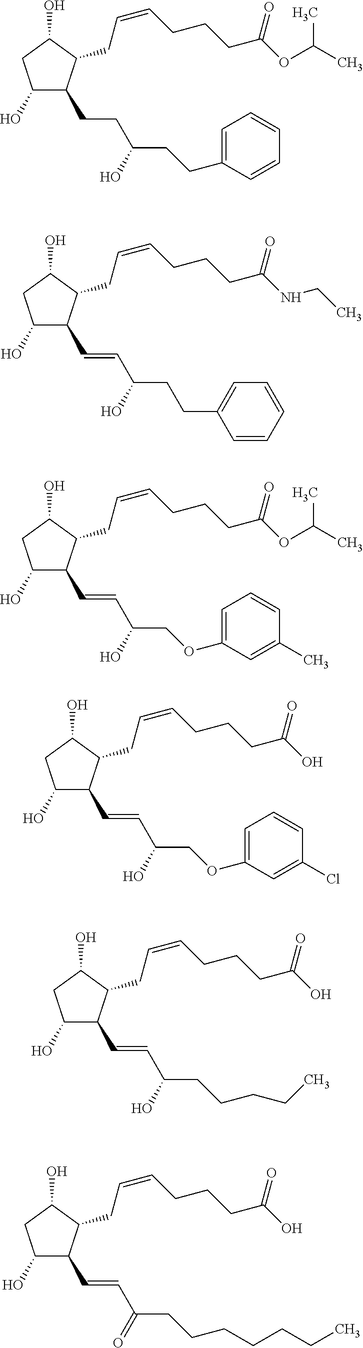

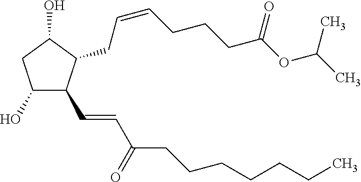

In a preferred embodiment, the present invention provides a process for the production of Latanoprost, Bimatoprost and Travoprost as mentioned below in scheme 2 and scheme 3.

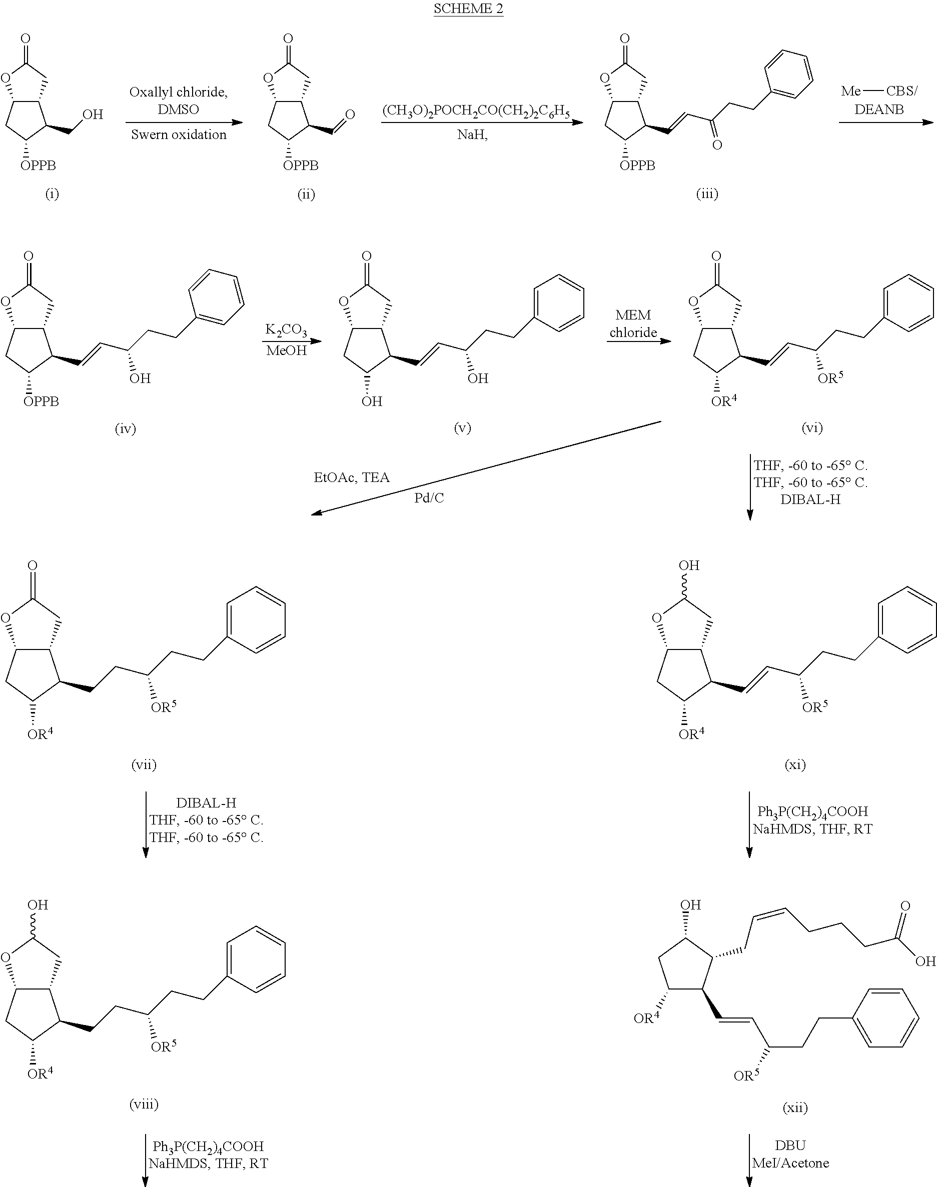

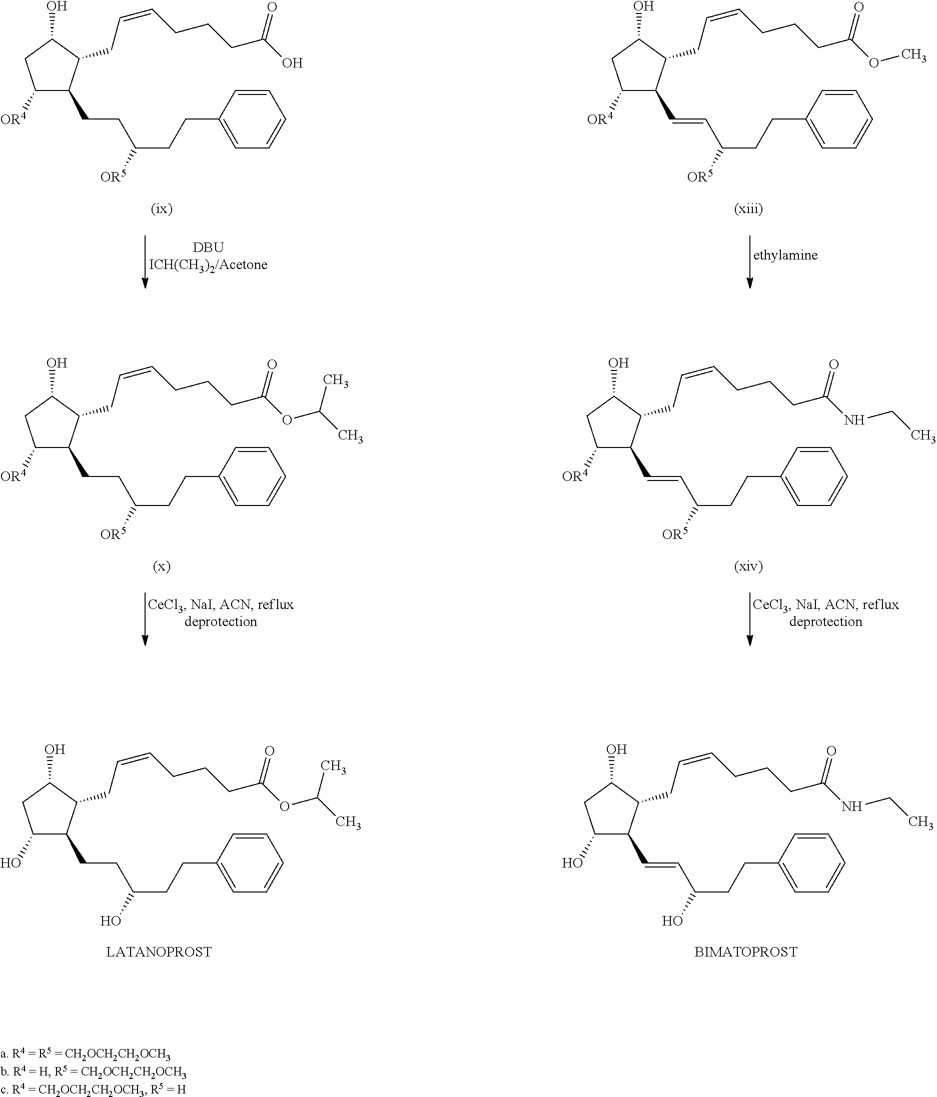

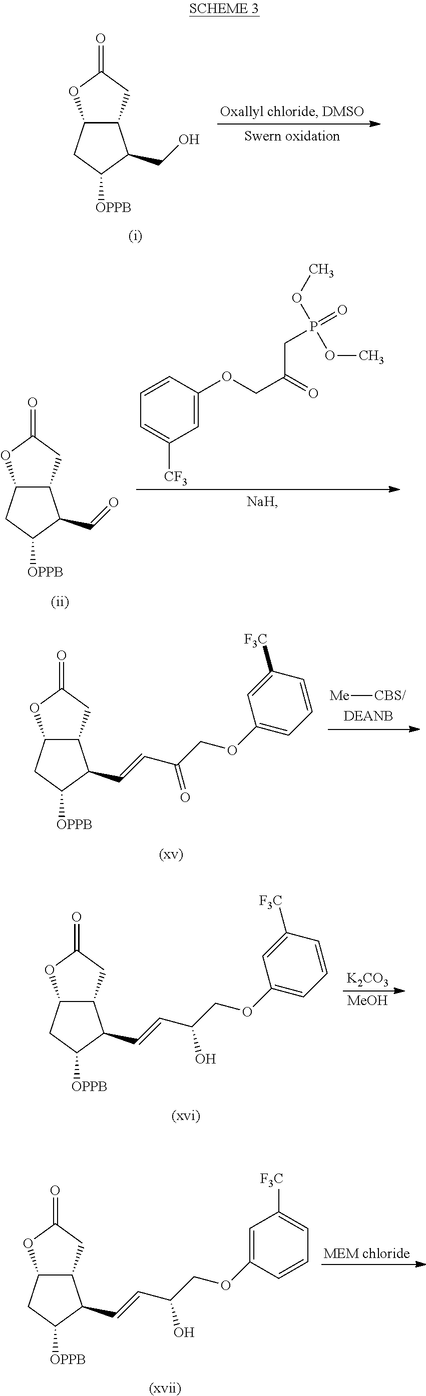

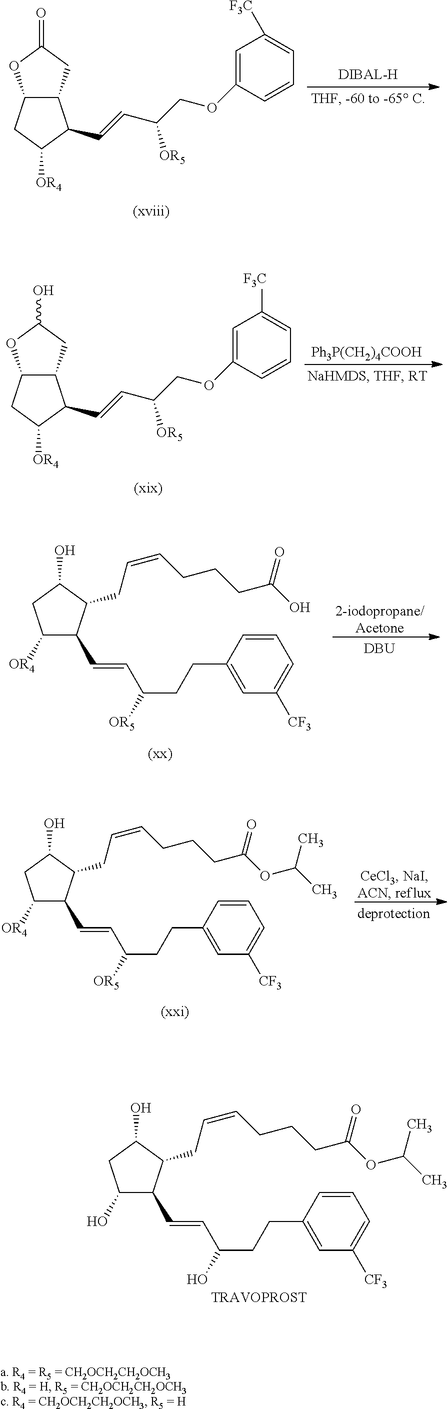

### DEFINITIONS AND CONVENTIONS

The definitions and explanations below are for the terms as used throughout this entire document including both the specification and the claims.

### DEFINITIONS

All temperatures are in degrees Celsius.

MTBE refers to methyl t-butyl ether.
TLC refers to thin-layer chromatography.
THF refers to tetrahydrofuran.
THP refers to tetrahydropyranyl.
NaHMDS refers to sodium hexamethyldisilazane.
MEM Chloride refers to 2-methoxyethoxymethyl chloride.
DIBAL-H refers to disiobutylaluminium hydride.
DBU refers to 1,8-Diazabicyclo[5.4.0]undec-7-ene.
(R)—N-MeCBS refers to (R)-methyl oxazaborolidine in toluene (1 M solution).
RT refers to room temperature
ACN refers to acetonitrile.
CeC3 refers to Cerium chloride
NaI refers to sodium Iodide.
g refers to gram
v refers to volume
h refers to hours
DMSO refers to dimethylsulphoxide
DEANB refers to Borane N,N′-diethylaniline complex
Chromatography (column and flash chromatography) refers to purification/separation of compounds. It is understood that the appropriate fractions are pooled and concentrated to give the desired compound(s).

The technology of the instant Application is further elaborated with the help of following examples. However, the examples should not be construed to limit the scope of the invention.

### Example 1

### Preparation of (3aR,4R,6aS)-4-formyl-2-oxohexahydro-2H-cyclopenta[b]furan-5-yl biphenyl-4-carboxylate (compound-ii)

A solution of oxalyl chloride (50 mL) in dry dichloromethane (7.5 L) was chilled to −70° C. under nitrogen atmosphere. Added slowly dimethylsulphoxide (DMSO) (44.3 g, 0.568 moles) to the reaction mixture at −70° C. and stirred at that temperature for 30 minutes. Then added Corey lactone (compound (i)) (50 g) dissolved in dichloromethane (500 mL) to the reaction mixture at −70° C. and stirred for one hour at that temperature. Then added triethylamine (72 g) and stirred for one hour. The reaction completion was monitored by TLC. After the reaction completion, the mass was quenched with saturated ammonium chloride solution (50 mL). The organic layer was separated, washed with saturated sodium chloride solution and dried over sodium sulphate. The organic layer was filtered and carried the organic layer (compound (II)) immediately to the next step.

### Example 2

### Preparation of (3aR,4R,6aS)-2-oxo-4-((E)-3-oxo-5-phenylpent-1-enyl)hexahydro-2H-cyclopenta[b]furan-5-yl biphenyl-4-carboxylate (compound-iii)

A solution of dimethyl (2-oxo-4-phenylbutyl)phosphonate (34 g) in dichloromethane (1 L) was added drop wise at 0° C. to a suspension of 60° A sodium hydride (5.7 g) in dichloromethane (1 L). The mixture was stirred at 0° C. for one hour. The solution of aldehyde (compound (II)) (50 g) in dichloromethane prepared in the previous stage was added drop wise to the mixture at 0-5° C. The reaction completion was monitored by TLC. After the reaction completion, filtered the reaction mass over celite bed and filtrate was washed with water (1 L), saturated sodium chloride solution (1 L). The organic layer was dried over sodium sulphate, filtered and concentrated under reduced pressure to residue. The crude brown residue (compound (iii) (65 g) was taken for the next step.

### Example 3

### Preparation of (3aR,4R,6aS)-4-((S,E)-3-hydroxy-5-phenylpent-1-enyl)-2-oxohexahydro-2H-cyclopenta[b]furan-5-yl biphenyl-4-carboxylate (compound-iv)

A 1M solution of (R)—N-MeCBS (14 mL) was added drop wise at 25° C. to the solution of compound (iii) (65 g) in THF (2 L) under nitrogen atmosphere. Stirred for 30 minutes at 25° C., then chilled the reaction mixture to −20° C., added Borane N,N′-diethylaniline complex (DEANB) (11.1 mL) at −20° C. and slowly raised the temperature to 0° C. The reaction completion was monitored by TLC. After the reaction completion, quenched the reaction mass with methanol (130 mL) and acidified to pH 3 using 1.5 N HCl (130 mL). Added ethylacetate (1 L) and water (500 mL), the organic layer was separated, washed with water (500 mL), saturated sodium chloride solution (250 mL) and dried over sodium sulphate. The organic layer was concentrated under reduced pressure to residue. The brown residue was purified by flash column chromatography to separate the desired isomer to obtain the title compound (iv) as white solid (20 g).

### Example 4

### Preparation of (3aR,4R,5R,6aS)-5-hydroxy-4-[(1E,3S)-3-hydroxy-5-phenylpent-1-en-1-yl]hexahydro-2H-cyclopenta[b]furan-2-one (compound-v)

Potassium carbonate (3.4 g) was added to a solution of compound (iv) (20 g) dissolved in methanol (200 mL). The reaction mixture was stirred at 25° C. for 2 h. The reaction completion was monitored by TLC. After the reaction completion pH of the mass was adjusted to 6 using 1.5 N HCl. The reaction mass was concentrated to remove methanol. Added ethylacetate (500 mL) and water (250 mL), stirred for 5 minutes and the layers were separated. the organic layer was washed with saturated sodium chloride solution and dried over sodium sulphate. The organic layer was concentrated under reduced pressure to residue. The yellow oil obtained was purified by column chromatography to get compound (v) (light yellow oil) (10 g).

### Example 5

### Preparation of (3aR,4R,5R,6aS)-5-((2-methoxyethoxy)methoxy)-4-((R)-3-((2-methoxyethoxy)methoxy)-5-phenylpentyl)hexahydro-2H-cyclopenta[b]furan-2-one (compound-vi)

A solution of compound (v) (10 g) in dry dichloromethane (210 mL) was chilled to 0° C. under nitrogen blanket. Added diisopropylethylamine (31.3 mL) and methoxyethoxymethyl chloride (MEM chloride) (15 mL) to the reaction mixture at 0° C. Then slowly raised the temperature to 25° C., stirred for 8 hours at that temperature. The reaction completion was monitored by TLC. After the reaction completion, the mass was quenched with water (100 mL). The layers were separated and the organic layer was washed with saturated sodium chloride solution (50 mL) and dried over sodiumsulphate. The organic layer was concentrated under reduced pressure to syrup stage (12 g). The brown syrup (compound (vi)) was taken to next stage without purification.

### Example 6

### Preparation of (3aR,4R,5R,6aS)-5-((2-methoxyethoxy)methoxy)-4-((R)-3-((2-methoxyethoxy)methoxy)-5-phenylpentyl)hexahydro-2H-cyclopenta[b]furan-2-one (compound-vii)

Added palladium on carbon (1.2 g, 50% wet) to a solution of compound (vi) in ethylacetate (120 mL). Added triethylamine (1.2 mL) and stirred under Hydrogen pressure (2 Kg/Cm²) at 25° C. for 2 h. the reaction completion was monitored by TLC. After the reaction completion the mass was filtered over celite bed. The clear filtrate was concentrated under reduced pressure to syrup. The syrup (compound (viii)) thus obtained was taken to nest stage without purification.

### Example 7

### Preparation of (3aR,4R,5R,6aS)-5-((2-methoxyethoxy)methoxy)-4-((R)-3-((2-methoxyethoxy)methoxy)-5-phenylpentyl)hexahydro-2H-cyclopenta[b]furan-2-ol (compound-viii)

A solution of compound (vii) (12 g) in dry toluene (360 mL) was cooled to −65° C. under nitrogen atmosphere. Added DIBAL-H (75 mL) drop wise at −60° C. to −65° C. The reaction mass was stirred at −60° C. to −65° C. for one hour and reaction completion was monitored by TLC. After reaction completion, the mass was quenched with methanol (112.5 mL) and stirred at 25° C. for one hour. The mass was filtered over celite bed and the filtrate was concentrated under reduced pressure to reside. The residue was dissolved in ethyl acetate (500 mL), added water (250 mL), stirred and the layers were separated. The ethyl acetate layer was washed with saturated sodium chloride solution (50 mL), dried over sodium sulphate and filtered. The organic layer was concentrated under reduced pressure to syrup stage (10 g). The syrup (compound (viii) thus obtained was taken to next step without purification.

### Example 8

### Preparation of (Z)-7-(1R,2R,3R,5S)-5-hydroxy-3-((2-methoxyethoxy)methoxy)-2-((R)-3-((2-methoxyethoxy)methoxy)-5-phenylpentyl)cyclopentyl)hept-5-enoic acid (compound-ix)

1M solution of NaHMDS (156 mL) was added to the suspension of (4-carboxy butyl) triphenyl phosphonium bromide (35 g) in dry THF (350 mL) under nitrogen atmosphere at 25° C. The solution turned orange and mixture was stirred for 30 minutes. Added compound (viii) (10 g) dissolved in dry THF (20 mL) to the reaction mixture, the solution turned brownish. The reaction mass was stirred at 25° C. for 1.5 h and the reaction completion was monitored by TLC. After the reaction completion, the mass was quenched with water (1 L) and the layer was washed with ethyl acetate (200 mL). The pH of aqueous layer was adjusted to 5 by using 2° A citric acid solution (100 mL) and product was extracted to ethyl actate (2×500 mL). The combined organic layer was washed with saturated sodium chloride solution (100 mL) and dried over sodiumsulphate. The layer was filtered and concentrated under reduced pressure to get brown syrup (compound (ix)) (25 g).

### Example 9

### Preparation of (Z)-isopropyl 7-((1R,2R,3R,5S)-5-hydroxy-3-((2-methoxyethoxy)methoxy)-2-((R)-3-((2-methoxyethoxy)methoxy)-5-phenylpentyl)cyclopentyl)hept-5-enoate (compound-x)

2-iodopropane (25 mL, 0.2470 moles) and DBU (37 mL) was added to stirred solution of compound (ix) (25 g) in dry acetone (500 mL) under nitrogen at 25° C. The mass was stirred at that temperature for 8 hours and the reaction completion was monitored by TLC. After the reaction completion the mass was concentrated under reduced pressure to remove acetone. Added 2% citric acid solution (250 mL) and the product was extracted to ethyl acetate (500 mL). The organic layer was washed with water (200 mL), saturated sodium chloride solution (100 mL) and dried over sodiumsulphate. The layer was filtered and concentrated under reduced pressure to syrup stage (26 g). The syrup (compound (x)) thus obtained was taken to next step without purification.

### Example 10

### Preparation of (Z)-isopropyl 7-((1R,2R,3R,5S)-3,5-dihydroxy-2-((R)-3-hydroxy-5-phenylpentyl)cyclopentyl)hept-5-enoate (Latanoprost)

Added cerium (III) chloride heptahydrate (16 g) and sodium iodide (4 g) to solution of compound (x) (26 g) in acetonitrile (260 mL) and contents were refluxed at 80-85° C. for 2 h. The reaction completion was monitored by TLC. After the reaction completion the mass was concentrated under reduced pressure to remove acetonitrile. The residue was diluted with water (200 mL) and the product was extracted with ethyl acetate (500 mL). The ethyl acetate layer was washed with saturate sodium chloride solution (100 mL), dried over sodium sulphate and filtered. The layer was concentrated under reduced pressure to obtain crude Latanoprost. The crude Latanoprost was purified by column chromatograph and was further purified by prep HPLC to obtain Latanoprost of purity greater than 99% (4 g).

### Example 11

### Preparation of (3aR,4R,5R,6aS)-4-((S,E)-3-((2-methoxyethoxy)methoxy)-5-phenylpent-1-enyl)hexahydro-2H-cyclopenta[b]furan-2,5-diol (compound-xi)

A solution of compound (vii) with mono MEM protected [(3aR,4R,5R,6aS)-5-hydroxy-4-((S,E)-3-((2-methoxyethoxy)methoxy)-5-phenylpent-1-enyl)hexahydro-2H-cyclopenta[b]furan-2-one] (12 g, 0.0307 moles) in dry toluene (360 mL) was cooled to −65° C. under nitrogen atmosphere. Added DIBAL-H (92 mL) drop wise at −60° C. to −65° C. The reaction mass was stirred at −60° C. to −65° C. for one hour and reaction completion was monitored by TLC. After reaction completion, the mass was quenched with methanol (112.5 mL) and stirred at 25° C. for one hour. The mass was filtered over celite bed and the filtrate was concentrated under reduced pressure to reside. The residue was dissolved in ethyl acetate (500 mL), added water (250 mL), stirred and the layers were separated. The ethyl acetate layer was washed with saturated sodium chloride solution (50 mL), dried over sodium sulphate and filtered. The organic layer was concentrated under reduced pressure to syrup stage (10 g). The syrup (compound (viii) thus obtained was taken to next step without purification.

### Example 12

### Preparation of (Z)-methyl 7-((1R,2R,3R,5S)-3,5-dihydroxy-2-((S,E)-3-((2-methoxyethoxy)methoxy)-5-phenylpent-1-enyl)cyclopentyl)hept-5-enoate (compound-xii)

1M solution of NaHMDS (193 mL) was added to the suspension of (4-carboxy butyl) triphenyl phosphonium bromide (43 g) in dry THF (350 mL) under nitrogen atmosphere at 25° C. The solution turned orange and mixture was stirred for 30 minutes. Added compound (viii) (10 g) dissolved in dry THF (20 mL) to the reaction mixture, the solution turned brownish. The reaction mass was stirred at 25° C. for 1.5 h and the reaction completion was monitored by TLC. After the reaction completion, the mass was quenched with water (1 L) and the layer was washed with ethyl acetate (200 mL). The pH of aqueous layer was adjusted to 5 by using 2% citric acid solution (100 mL) and product was extracted to ethyl actate (2×500 mL). The combined organic layer was washed with saturated sodium chloride solution (100 mL) and dried over sodiumsulphate. The layer was filtered and concentrated under reduced pressure to get brown syrup (compound (ix)) (25 g).

### Example 13

### Preparation of (Z)-methyl 7-((1R,2R,3R,5S)-3,5-dihydroxy-2-((S,E)-3-((2-methoxyethoxy)methoxy)-5-phenylpent-1-enyl)cyclopentyl)hept-5-enoate (compound-xiii)

Methyl iodide (18.3 mL) and DBU (44 mL) was added to stirred solution of compound (ix) (25 g) in dry acetone (500 mL) under nitrogen at 25° C. The mass was stirred at that temperature for 8 hours and the reaction completion was monitored by TLC. After the reaction completion the mass was concentrated under reduced pressure to remove acetone. Added 2° A citric acid solution (250 mL) and the product was extracted to ethyl acetate (500 mL). The organic layer was washed with water (200 mL), saturated sodium chloride solution (100 mL) and dried over sodiumsulphate. The layer was filtered and concentrated under reduced pressure to syrup stage (26 g). The syrup (compound (x)) thus obtained was taken to next step without purification.

### Example 14

### Preparation of (Z)-7-((1R,2R,3R,5S)-3,5-dihydroxy-2-((S,E)-3-((2-methoxyethoxy)methoxy)-5-phenylpent-1-enyl)cyclopentyl)-N-ethylhept-5-enamide (compound-xiv)

Aqueous ethylamine (70%, 1 L, 40 v) was added to crude compound (xiii) and stirred for 24 h at 25° C. The reaction completion was monitored by TLC. After the reaction completion, the mass was neutralised by using sodium hydrogen sulphate solution (5% solution). The product was extracted to dichloromethane (500 mL) and the organic layer was washed with water (200 mL), saturated sodium chloride solution (50 mL) and dried over sodiumsulphate. The layer was filtered and concentrated to residue (26 g). The light brown solid (compound (xiv)) was taken for deprotection without purification.

### Example 15

### Preparation of (Z)-7-(1R,2R,3R,5S)-3,5-dihydroxy-2-((S,E)-3-hydroxy-5-phenylpent-1-enyl)cyclopentyl)-N-ethylhept-5-enamide (Bimatoprost)

Added cerium (III) chloride heptahydrate (16 g) and sodium iodide (4 g) to solution of compound (x) (26 g) in acetonitrile (260 mL) and contents were refluxed at 80-85° C. for 2 h. The reaction completion was monitored by TLC. After the reaction completion the mass was concentrated under reduced pressure to remove acetonitrile. The residue was diluted with water (200 mL) and the product was extracted with ethyl acetate (500 mL). the ethyl acetate layer was washed with saturate sodium chloride solution (100 mL), dried over sodium sulphate and filtered. The layer was concentrated under reduced pressure to obtain crude Bimatoprost. The crude Bimatoprost was purified by column chromatography method. The pure fractions from the column were pooled and concentrated to syrup stage and the product was crystallized by using diethyl ether. The product Bimatoprost obtained was of purity greater than 99% (4 g).

### Example 16

### Preparation of (3aR,4R,5R,6aS)-2-oxo-4-((E)-3-oxo-4-(3-(trifluoromethyl)phenoxy)but-1-enyl)hexahydro-2H-cyclopenta[b]furan-5-yl biphenyl-4-carboxylate (compound-xv)

A solution of dimethyl 2-oxo-3-(3-(trifluoromethyl)phenoxy)propylphosphonate (44 g) in dichloromethane (1 L) was added drop wise at 0° C. to a suspension of 60% sodium hydride (5.7 g) in dichloromethane (1 L). The mixture was stirred at 0° C. for one hour. The solution of aldehyde

(compound (II)) (50 g) in dichloromethane prepared in the previous stage was added drop wise to the mixture at 0-5° C. The reaction completion was monitored by TLC. After the reaction completion, filtered the reaction mass over celite bed and filtrate was washed with water (1 L), saturated sodium chloride solution (1 L). The organic layer was dried over sodium sulphate, filtered and concentrated under reduced pressure to residue. The crude brown residue (compound (xv) (61 g) was taken for the next step.

### Example 17

### Preparation of (3aR,4R,5R,6aS)-4-((R,E)-3-hydroxy-4-(3-(trifluoromethyl)phenoxy)but-1-enyl)-2-oxohexahydro-2H-cyclopenta[b]furan-5-yl biphenyl-4-carboxylate (compound-xvi)

A 1M solution of (R)—N-MeCBS (11 mL) was added drop wise at 25° C. to the solution of compound (xv) (61 g, 0.1107 moles) in THF (2 L) under nitrogen atmosphere. Stirred for 30 minutes at 25° C., then chilled the reaction mixture to −20° C., added Borane N,N′-diethylaniline complex (DEANB) (20 mL) at −20° C. and slowly raised the temperature to 0° C. The reaction completion was monitored by TLC. After the reaction completion, quenched the reaction mass with methanol (130 mL) and acidified to pH 3 using 1.5 N HCl (130 mL). Added ethylacetate (1 L) and water (500 mL), the organic layer was separated, washed with water (500 mL), saturated sodium chloride solution (250 mL) and dried over sodium sulphate. The organic layer was concentrated under reduced pressure to residue. The brown residue was purified by flash column chromatography to separate the desired isomer to obtain the title compound (xvi) light yellow liquid (23 g).

### Example 18

### Preparation of (3aR,4R,5R,6aS)-5-hydroxy-4-((R,E)-3-hydroxy-4-(3-(trifluoromethyl)phenoxy)but-1-enyl)hexahydro-2H-cyclopenta[b]furan-2-one (compound-xvii)

Potassium carbonate (3.4 g) was added to a solution of compound (xvi) (23 g) dissolved in methanol (200 mL). The reaction mixture was stirred at 25° C. for 2 h. The reaction completion was monitored by TLC. After the reaction completion pH of the mass was adjusted to 6 using 1.5 N HCl. The reaction mass was concentrated to remove methanol. Added ethylacetate (500 mL) and water (250 mL), stirred for 5 minutes and the layers were separated. The organic layer was washed with saturated sodium chloride solution and dried over sodium sulphate. The organic layer was concentrated under reduced pressure to residue. The yellow oil obtained was purified by column chromatography to get compound (xvii) (light yellow oil) (10 g).

### Example 19

### Preparation of (3aR,4R,5R,6aS)-5-((2-methoxyethoxy)methoxy)-4-((R,E)-3-(2-methoxyethoxy)methoxy)-4-(3-(trifluoromethyl)phenoxy)but-1-enyl)hexahydro-2H-cyclopenta[b]furan-2-one (compound-xviii)

A solution of compound (xvii) (10 g) in dry dichloromethane (210 mL) was chilled to 0° C. under nitrogen blanket. Added diisopropylethylamine (25.6 mL) and methoxyethoxymethyl chloride (MEM chloride) (13.8 mL) to the reaction mixture at 0° C. Then slowly raised the temperature to 25° C., stirred for 8 hours at that temperature. The reaction completion was monitored by TLC. After the reaction completion, the mass was quenched with water (100 mL). The layers were separated and the organic layer was washed with saturated sodium chloride solution (50 mL) and dried over sodiumsulphate. The organic layer was concentrated under reduced pressure to syrup stage (13 g). The light brown syrup (compound (xviii)) was taken to next stage without purification.

### Example 20

### Preparation of ((3aR,4R,5R,6aS)-5-((2-methoxyethoxy)methoxy)-4-((R,E)-3-(2-methoxyethoxy)methoxy)-4-(3-(trifluoromethyl)phenoxy)but-1-enyl)hexahydro-2H-cyclopenta[b]furan-2-ol (compound-xix)

A solution of compound (xviii) (13 g) in dry toluene (360 mL) was cooled to −65° C. under nitrogen atmosphere. Added DIBAL-H (71 mL) drop wise at −60° C. to −65° C. The reaction mass was stirred at −60° C. to −65° C. for one hour and reaction completion was monitored by TLC. After reaction completion, the mass was quenched with methanol (112.5 mL) and stirred at 25° C. for one hour. The mass was filtered over celite bed and the filtrate was concentrated under reduced pressure to reside. The residue was dissolved in ethyl acetate (500 mL), added water (250 mL), stirred and the layers were separated. The ethyl acetate layer was washed with saturated sodium chloride solution (50 mL), dried over sodium sulphate and filtered. The organic layer was concentrated under reduced pressure to syrup stage (12 g). The syrup (compound (xix) thus obtained was taken to next step without purification.

### Example 21

### Preparation of (Z)-7-((1R,2R,3R,5S)-5-hydroxy-3-((2-methoxyethoxy)methoxy)-2-((R,E)-3-((2-methoxyethoxy)methoxy)-4-(3-(trifluoromethyl)phenoxy)but-1-enyl)cyclopentyl)hept-5-enoic acid (compound-xx)

1M solution of NaHMDS (166 mL) was added to the suspension of (4-carboxy butyl) triphenyl phosphonium bromide (36.7 g) in dry THF (350 mL) under nitrogen atmosphere at 25° C. The solution turned orange and mixture was stirred for 30 minutes. Added compound (xix) (12 g, 0.0218 moles) dissolved in dry THF (20 mL) to the reaction mixture, the solution turned brownish. The reaction mass was stirred at 25° C. for 1.5 h and the reaction completion was monitored by TLC. After the reaction completion, the mass was quenched with water (1 L) and the layer was washed with ethyl acetate (200 mL). The pH of aqueous layer was adjusted to 5 by using 2% citric acid solution (100 mL) and product was extracted to ethyl actate (2×500 mL). The combined organic layer was washed with saturated sodium chloride solution (100 mL) and dried over sodiumsulphate. The layer was filtered and concentrated under reduced pressure to get brown syrup (compound (xx)) (28 g).

### Example 22

### Preparation of (Z)-isopropyl 7-((1R,2R,3R,5S)-5-hydroxy-3-((2-methoxyethoxy)methoxy)-2-((R,E)-3-((2-methoxyethoxy)methoxy)-4-(3-(trifluoromethyl)phenoxy)but-1-enyl)cyclopentyl)hept-5-enoate (compound-xxi)

2-iodopropane (25 mL, 0.2470 moles) and DBU (37 mL) was added to stirred solution of compound (ix) (28 g) in dry acetone (500 mL) under nitrogen at 25° C. The mass was stirred at that temperature for 8 hours and the reaction completion was monitored by TLC. After the reaction completion the mass was concentrated under reduced pressure to remove acetone. Added 2% citric acid solution (250 mL) and the product was extracted to ethyl acetate (500 mL). The organic layer was washed with water (200 mL), saturated sodium chloride solution (100 mL) and dried over sodiumsulphate. The layer was filtered and concentrated under reduced pressure to syrup stage (26 g). The syrup (compound (xxi)) thus obtained was taken to next step without purification.

### Example 23

### Preparation of (Z)-isopropyl 7-((1R,2R,3R,5S)-3,5-dihydroxy-2-((R,E)-3-hydroxy-4-(3-(trifluoromethyl)phenoxy)but-1-enyl)cyclopentyl)hept-5-enoate (Travoprost)

Added cerium (III) chloride heptahydrate (14.3 g) and sodium iodide (3.4 g) to solution of compound (x) (26 g in acetonitrile (260 mL) and contents were refluxed at 80-85° C. for 2 h. The reaction completion was monitored by TLC. After the reaction completion the mass was concentrated under reduced pressure to remove acetonitrile. The residue was diluted with water (200 mL) and the product was extracted with ethyl acetate (500 mL). The ethyl acetate layer was washed with saturate sodium chloride solution (100 mL), dried over sodium sulphate and filtered. The layer was concentrated under reduced pressure to obtain crude Travoprost. The crude Travoprost was purified by column chromatograph and was further purified by prep HPLC to obtain Travoprost of purity greater than 99% (5 g).

We claim:

1. A process for preparing compound of formula; 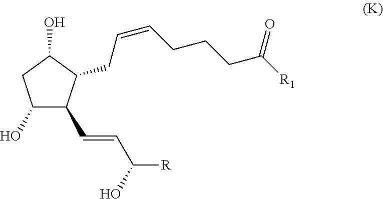
wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃; and R¹ is selected from OR³ and NHR³ wherein R³ is C₁-C₆ alkyl, H, and dashed lines ( [symbol] ) represents a double bond or a single bond comprises;
a). reacting compound of formula ‘I’ with haloalkane or ethylamine, 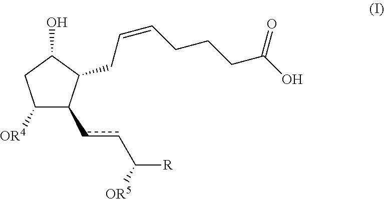
Wherein, R described as above, dashed line represents single or double bonds,
R⁴ and R⁵ represents, R⁴=R⁵=CH₂OCH₂CH₂CH₂CH₃; R⁴=H, R⁵=CH₂OCH₂CH₂CH₂CH₃; R⁴=CH₂OCH₂CH₂CH₂CH₃, R⁵=H to form compound of formula ‘J’ and, 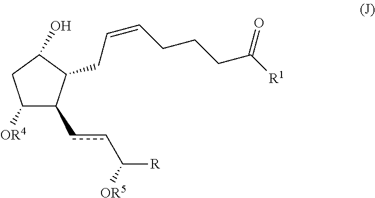
Wherein, R, R¹, dashed line, R⁴ and R⁵ as described above; and
b). deprotecting compound of formula ‘J’.

2. The process as claimed in claim 1, wherein said deprotection is done using cerium (III) chloride heptahydrate and sodium iodide in the presence of organic solvent.

3. The process as claimed in claim 2, wherein organic solvent is selected from a group comprising acetonitrile, ethanol, methanol, acetone and isopropyl alcohol.

4. The process as claimed in claim 1, wherein the compound K is any one of following compound;

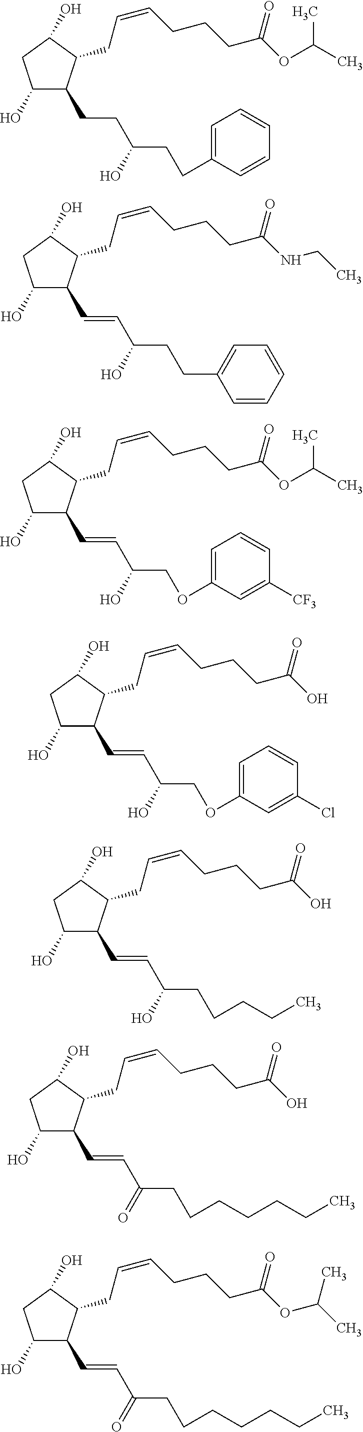

5. The process as claimed in claim 1, wherein process for the preparation of compound of formula ‘I’ comprises;
a). Reacting compound of formula ‘A’ 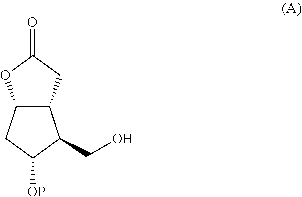
with dimethylsulphoxide, oxalylchloride and triethylamine in the presence of organic solvent to get compound of formula ‘B’, wherein P is selected from the group consisting of COX; in which X represents C₁ to C₆ alkyl, C₆-C₁₀ aryl which may be substituted or unsubstituted with one to three substituents independently selected from the group consisting of halo, C₁ to C₆ alkyl, unsubstituted C₆ to C₁₀ aryl, 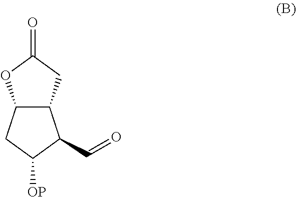
b). reacting compound of formula ‘B’ with 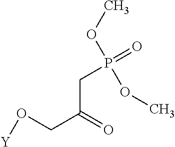
Wherein Y is selected from the group consisting of alkyl, aryl wherein aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃ in the presence of organic solvent to form compound of formula ‘C’ 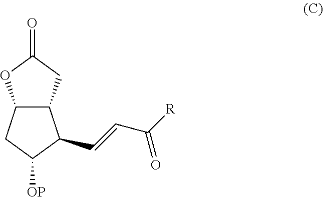
wherein R is selected from the group consisting of C₁-C₇ alkyl; C₇-C₁₇ aralkyl wherein the aryl group is unsubstituted or substituted with one to three substituents selected from the group consisting of C₁-C₆ alkyl, halo and CF₃; and (CH₂)ₙOR² wherein n is from 1 to 3 and R² represents a C₆-C₁₀ aryl group which is unsubstituted or substituted with one to three substituents selected from the group consisting of C₁-C₆ alkyl, halo and CF₃ and P is as described above,
c). by selective reduction of compound ‘C’ using Borane N,N′-diethylaniline complex in the presence of Corey catalyst to compound ‘D’, 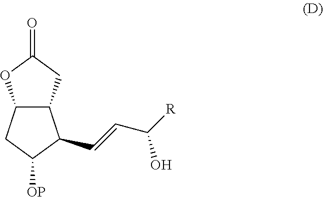
Wherein P and R are as described above,
d). deprotecting compound of formula D using base in organic solvent to get
compound of formula E, 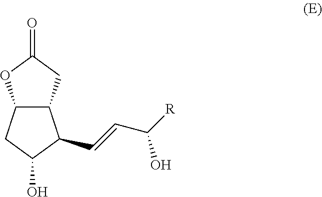
e). hydroxyl groups of compound E further protected to form compound of formula F, 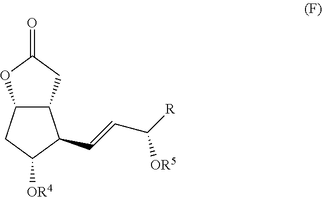
Wherein R described as above, R⁴ and R⁵ represents,
R⁴=R⁵=CH₂OCH₂CH₂CH₂CH₃; R⁴=H, R⁵=CH₂OCH₂CH₂CH₂CH₃;
R⁴=CH₂OCH₂CH₂CH₂CH₃, R⁵=H
f). compound of formula F optionally hydrogenated using palladium on carbon in the presence of organic solvent, further reducing the oxo group of this compound reduced to compound of formula H using DIBAL-H in the presence of organic solvent, 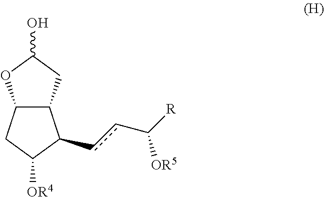
wherein the dashed line represents a double bond or a single bond; R, R⁴ and R⁵ represents as described above
g). compound of formula H further reacted with compound of formula PPh₃(CH₂)₄COOH in the presence of NaHMDS in organic solvent

6. The process as claimed in claim 5, wherein organic solvent is selected from a group comprising of alcohols, esters, tetrahydrofuran, pet ether, hexane, acetone and acetonitrile.

7. The process as claimed in claim 6, wherein said alcohols are selected from C₁ to C₄ alcohols.

8. The process as claimed in claim 6, wherein said esters are selected from ethyl acetate or butyl acetate.

9. The process as claimed in claim 5, wherein base is selected from potassium carbonate, sodium carbonate or sodium bi carbonate.

10. A compound of formula; 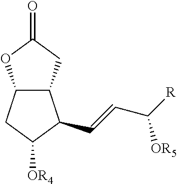
Wherein R described as above, R⁴ and R⁵ represents,
R⁴=R⁵=CH₂OCH₂CH₂CH₂CH₃; R⁴=H, R⁵=CH₂OCH₂CH₂CH₂CH₃;
R⁴=CH₂OCH₂CH₂CH₂CH₃, R⁵=H

11. A compound of formula; 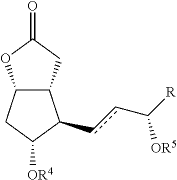
Wherein R described as above, dashed lines represents single or double bonds, R⁴ and R⁵ represents, R⁴=R⁵=CH₂OCH₂CH₂CH₂CH₃; R⁴=H,
R⁵=CH₂OCH₂CH₂CH₂CH₃; R⁴=CH₂OCH₂CH₂CH₂CH₃, R⁵=H

12. A compound of formula; 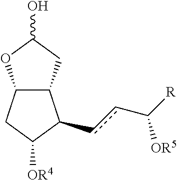
Wherein R described as above, dashed lines represents single or double bonds, R⁴ and R⁵ represents, R⁴=R⁵=CH₂OCH₂CH₂CH₂CH₃; R⁴=H,
R⁵=CH₂OCH₂CH₂CH₂CH₃; R⁴=CH₂OCH₂CH₂CH₂CH₃, R⁵=H

13. A compound of formula; 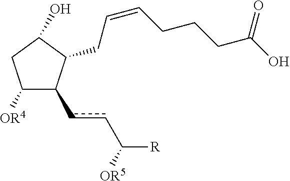
Wherein R described as above, dashed lines represents single or double bonds, R⁴ and R⁵ represents, R⁴=R⁵=CH₂OCH₂CH₂CH₂CH₃; R⁴=H,
R⁵=CH₂OCH₂CH₂ CH₂CH₃; R⁴=CH₂OCH₂CH₂ CH₂CH₃, R⁵=H

14. A compound 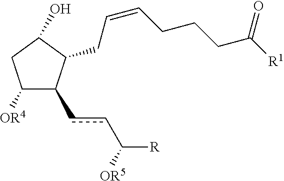
Wherein R described as above, dashed lines represents single or double bonds, R⁴ and R⁵ represents, R⁴=R⁵=CH₂OCH₂CH₂CH₂CH₃; R⁴=H,
R⁵=CH₂OCH₂CH₂CH₂CH₃; R⁴=CH₂OCH₂CH₂CH₂CH₃, R⁵=H
